(19) United States
(12) Patent Application Publication
(10) Pub. No.: US 2009/0325275 A1
(43) Pub. Date: Dec. 31, 2009

(54) CELL MANIPULATION AND CULTIVATION EQUIPMENTS FOR THE PRODUCTION OF CELL THERAPY PRODUCTS

(76) Inventors: Jeong-Yong Choi, Gyeonggi-do (KR); Dong-Sam Suh, Seoul (KR); Chang-Kwon Ko, Seoul (KR); Seung-Ju Ryu, Gyeonggi-do (KR); Hyang-Soon Chang, Seoul (KR); Jin-Wook Chung, Seoul (KR); Cheong-Ho Chang, Seoul (KR)

Correspondence Address: GWIPS;Peter T. Kwon, Gwacheon P.O. Box 72, 119 Byeolyang Ro, Gwacheon City, Gyeonggi-Do, 427-600, KR

(21) Appl. No.: 12/309,868

(22) PCT Filed: Feb. 15, 2007
(86) PCT No.: PCT/KR2007/000807

§ 371 Date: Feb. 2, 2009

(30) Foreign Application Priority Data
Aug. 11, 2006 (KR) 10-2006-0076276

(51) Int. Cl. C12M 1/18

(52) U.S. Cl. 435/287.1

(57) ABSTRACT

Cell manipulation and cultivation equipment for the production of cell therapy products has developed for preventing contamination of cells and improving the utility of a space. The equipment includes a room for the production and testing of cell therapy products, the room having an inner space enclosed in all sides and an outer entrance/exit door provided at any one side thereof, an L-shaped partition installed in a position of the room for the production and testing of cell therapy products, the partition having an inner entrance/exit door provided at any one side thereof, and a clean bench device placed inside the partition, the clean bench device including first, second, and third clean benches for preventing contamination of cells and increasing the utility of a space to the maximum extent. The operating of the clean room and clean bench has advantages to prevent contamination and improving the quality and reliability of cell therapy products.

### BACKGROUND OF THE INVENTION

1. Field of the Invention

The present invention relates to equipment for manipulation and cultivation of cells, for the production of cell therapy products. More particularly, the present invention relates to cell manipulation and cultivation equipment which can provide the advantages of a remarkably improved clean operating room or clean bench, enhanced space utility, agreeable operating environment, and convenience of use via additional convenience facilities, thereby preventing contamination of cells during the cultivation and production of cell therapy products, and achieving a great improvement in the quality and reliability of cell therapy products, so as to give a good image to consumers.

2. Related Prior Art

As well known, a clean bench is designed to basically prevent or intercept the leakage of harmful bacteria or materials, for the sake of safety of operators handling the harmful bacteria or materials. A great number of patents related to the clean bench have filed and registered.

One example of the patents includes Korean Patent Application No. 1998-0032987 (Korean Patent Registration No. 0308666) entitled “Clean bench”.

Now, the configuration of a conventional clean bench disclosed in the above patent will be described.

FIG. 1 is a block diagram illustrating the operation of a clean bench 1 employing a remote-control manipulation. As shown in FIGS. 1 and 2, the disclosed clean bench 1 includes: an electric motor 22 for generating air stream via rotation thereof and blowing the air stream to a sterilizing filter 80, under the control of a controller 21; the sterilizing filter 80 for sterilizing the air blown by the motor 22; a remote-control signal transmitter 10 for generating an input control signal in response to the operator's manipulation of a remote control 2; a remote-control signal receiver 20 for receiving the input control signal from the remote-control signal transmitter 10 integrated in the remote control 2; a timer 30 for regulating a sterilizing time, etc. under the control of the controller 21; a fluorescent light 40 for illuminating an operating space; an ultraviolet (UV) sterilizing light 50; and a buzzer 60 for generating an alarm sound informing the need to exchange a filter. Hem the controller 21 controls the overall operation of the motor 22 and the timer 30, for example, revolutions per minute of the motor 22, on the basis of the input control signal from the remote-control signal receiver 20.

The conventional clean bench having the above described configuration operates as follows.

If the remote-control signal transmitter generates an input control signal by the operator's manipulation of the remote control 2, the remote-control signal receiver 20 located in the clean bench 1 receives the input control signal, and in turn, the input control signal received by the remote-control signal receiver 20 is transmitted to the controller 21. Thereby, the controller 21 controls the operation of the motor 22, timer 30, etc. on the basis of the input control signal transmitted thereto, so as to enable the On-Off operation of a power switch of the clean bench 1, the flow rate of air, the On-Off operation of the fluorescent light and UV sterilizing light, the reservation of a sterilizing time, etc. to be regulated at a remote distance by use of the remote control 2.

However, the above described conventional clean bench has the following problems.

For example, the conventional clean bench has no inventive technical effect except for the fact that it can be manipulated at a remote distance by use of the remote control 2. In other words, the conventional clean bench has no function of preventing contamination of the clean bench, providing efficient layout of cell cultivation facilities within a small space for the sake of improving the utility of space, or fulfilling appropriate cell cultivation conditions, etc.

### SUMMARY OF THE INVENTION

Therefore, the present invention has been made in view of the above problems, and it is a first object of the present invention to provide a clean bench device within an operating room for the production and testing of cell therapy products. It is a second object of the present invention to facilitate the construction of a clean room even in a small laboratory or building by integrating a variety of tools and utensils and arranging them to achieve the operator's optimum movement path. In particular, when the clean bench device has a U-shaped arrangement, the clean bench can achieve an increased operating space required for the cultivation of cells and the production of cell therapy products even if the clean bench device is mounted in a small clean room. In addition, when the clean bench device includes first, second, and third clean benches and cell cultivation equipment and tools are disposed at their right places, it is possible to reduce the operator's unnecessary movement and to separately perform an operation each clean bench according to works which might cause the possibility of contamination. This has the effect of minimizing the possibility of contamination and remarkably improving the clean room and clean benches. It is a third object of the present invention to provide a clean bench, to which a transparent plate, fluorescent light, UV sterilizing light, computer monitor, incubator, etc. are integrally installed, so as to improve the operation environment and the utility of a space. It is a fourth object of the present invention to provide a clean bench, to which a variety of convenience devices, such as an operating panel, foot switch, foldable drawer, etc., are added, so as to achieve convenience of use. It is a fifth object of the present invention to prevent contamination of cell manipulation and cultivation equipment for the production of cell therapy products. It is a sixth object of the present invention to provide cell manipulation and cultivation equipment for the production of cell therapy products, which can achieve a remarkable improvement in the quality and reliability of the cell therapy products, so as to give a good image to consumers.

In accordance with an aspect of the present invention, the above and other objects can be accomplished by the provision of cell manipulation and cultivation equipment for the production of cell therapy products comprising: a room for the production and testing of cell therapy products, the room having an inner space enclosed in all sides and an outer entrance/exit door provided at any one side thereof; an L-shaped partition installed in a position of the room for the production and testing of cell therapy products, the partition having an inner entrance/exit door provided at any one side thereof; and a clean bench device placed inside the partition, the clean bench device including first, second, and third clean benches for preventing contamination of cells and increasing the utility of a space to the maximum extent.

The present invention provides a clean bench device within an operating room for the production and testing of cell therapy products, to prevent contamination of the clean bench device while increasing the utility of a space to the maximum extent. For this, a variety of tools and utensils, provided at the clean bench device, are integrally formed and arranged to achieve the operator's optimum movement path. Thereby, it is possible to facilitate the construction of a clean room even in a small laboratory or building.

In particular, according to the present invention, the clean bench device has a U-shaped arrangement, to achieve an increased operating space required for the cultivation of cells and the production of cell therapy products even if the clean bench device is mounted in a small clean room. In addition, the clean bench device includes first, second, and third clean benches and cell cultivation equipment and tools are disposed at their right places. As a result, it is possible to reduce the operator's unnecessary movement and to separately perform an operation per each clean bench according to the possibility of contamination of each clean bench. This has the effect of minimizing the possibility of contamination and remarkably improving the clean room and clean benches.

In the present invention, the clean bench is integrally provided with a transparent plate, fluorescent light, UV sterilizing light, computer monitor, incubator, etc., so as to improve the environment of an operation and the utility of a space.

The clean bench is further provided with a variety of convenience devices, such as an operating panel, foot switch, foldable drawer, etc., so as to achieve convenience of use.

Consequently, it can be said that the present invention is a very useful invention capable of achieving a remarkable improvement in the quality and reliability of the cell therapy products, so as to give a good image to consumers

### DESCRIPTION OF THE DRAWINGS

FIG. 1 is a block diagram of a conventional clean bench;

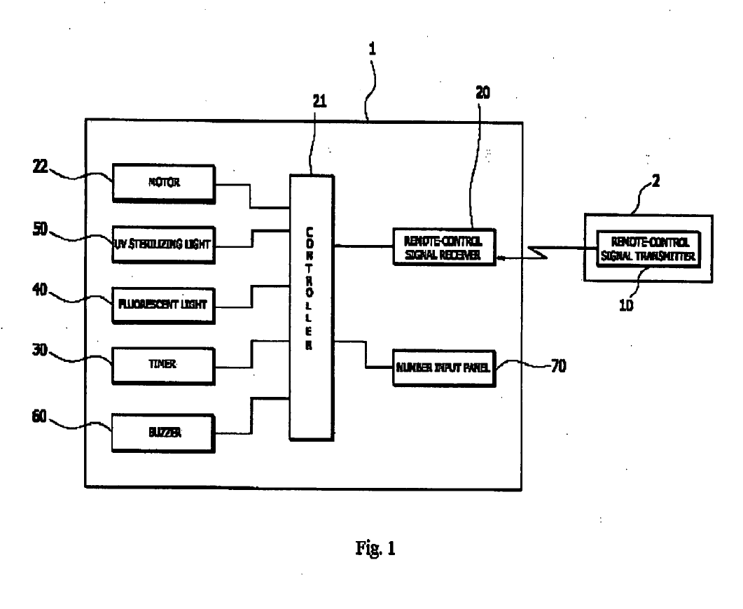

FIG. 2 is a schematic elevation view illustrating a sterilizing course of the conventional clean bench;

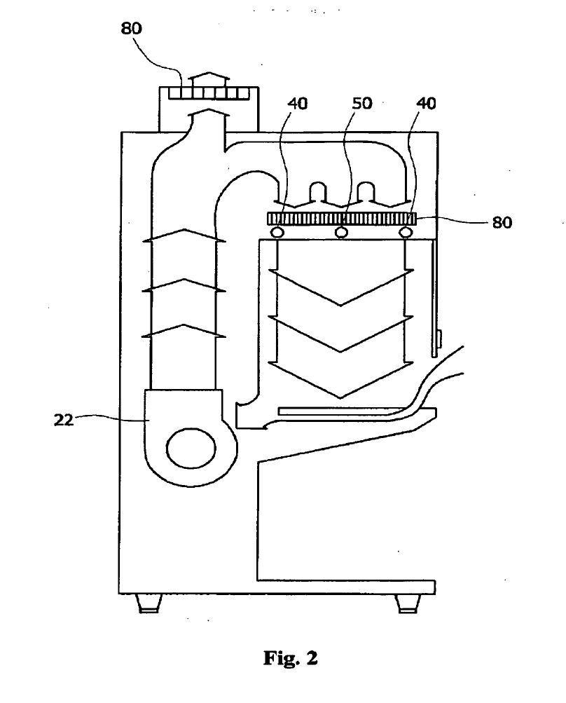

FIG. 3 is a plan view illustrating the overall configuration of cell manipulation and cultivation equipment for the production of cell therapy products according to the present invention;

FIG. 4 is a perspective view illustrating the important parts of the cell manipulation and cultivation equipment for the production of cell therapy products according to the present invention;

FIG. 5 is a front view illustrating a first clean bench according to the present invention;

FIG. 6 is a front view illustrating a second clean bench according to the present invention;

FIG. 7 is a side sectional view of the second clean bench according to the present invention; and

FIG. 8 is a front view illustrating a third clean bench according to the present invention.

### DETAILED DESCRIPTION OF THE PREFERRED EMBODIMENTS

Hereinafter, preferred embodiments of cell manipulation and cultivation equipment according to the present invention for achieving the above described objects will be described in detail with reference to the accompanying drawings.

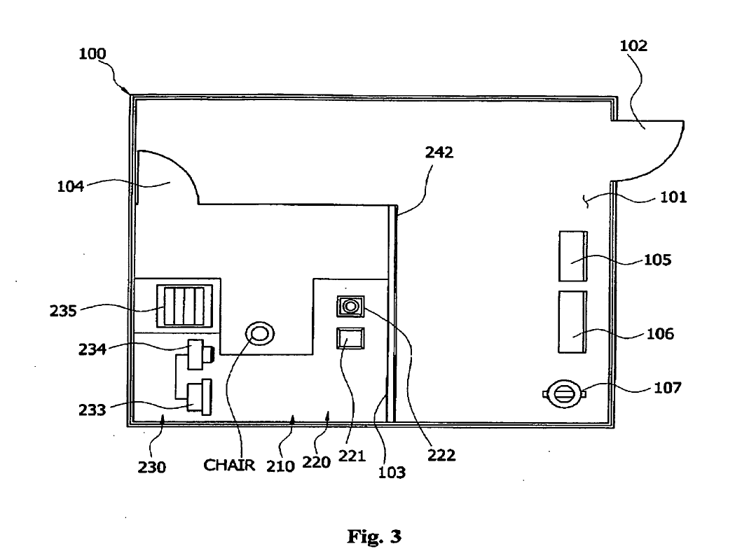

The cell manipulation and cultivation equipment for the production of cell therapy products has a configuration as shown in FIGS. 3 to 8.

In the following description of the present invention, detailed description related to known functions or configurations will be omitted if it is judged that the detailed description unnecessary makes the subject matter of the present invention unclear.

In addition, the terms, which will be described hereinafter, are determined in consideration of functions of corresponding elements in the present invention, and may be changed according to the manufacture's intention or custom. Accordingly, the definition of the terms has to be based on the overall description of herein.

Referring first to FIG. 3, the cell manipulation and cultivation equipment according to the present invention basically comprises: a room 100 for the production and testing of cell therapy products, the room 100 having an inner space 101 enclosed in all sides and an outer entrance/exit door 102 provided at any one side thereof; an L-shaped partition 103 installed in a position of the room 100 for the production and testing of cell therapy products, the partition 103 having an inner entrance/exit door 104 provided at any one side thereof; and a clean bench device 200 placed inside the partition 103, the clean bench device 200 including first, second, and third clean benches 210, 220, and 230, for preventing contamination of cells and increasing the utility of a space to the maximum extent.

In the cell manipulation and cultivation equipment according to the present invention, a freezer 105, a refrigerator 106 and a liquid nitrogen tank 107 are placed in the space 101 of the room 100 outside of the clean bench device 200, to prevent heat emitted from the freezer and refrigerator from entering the clean benches and to reduce the size of the clean benches.

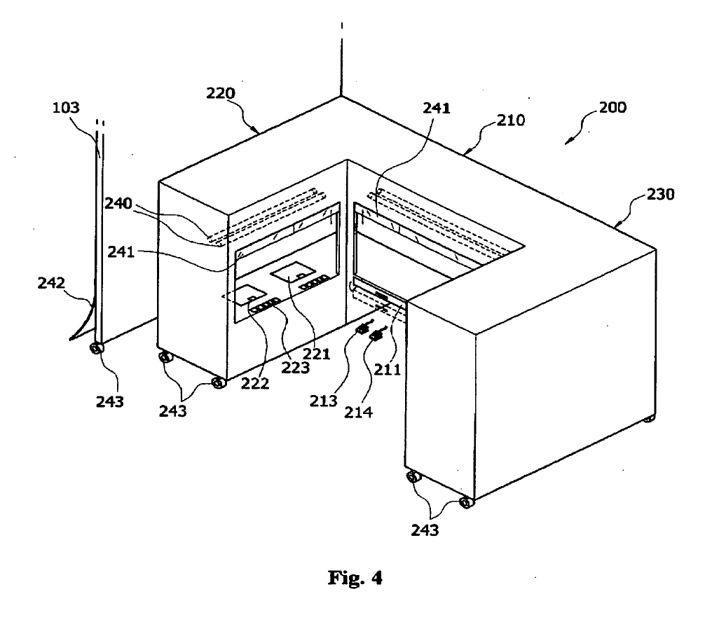

The first, second, and third clean benches 210, 220, and 230 according to the present invention, as shown in FIGS. 3 and 4, have a U-shaped arrangement such that the second and third clean benches 220 and 230 are located at the left and right sides of the first clean bench 210. This U-shaped arrangement is efficient to increase the overall space of the clean benches, thereby achieving a sufficient operating space, required for the cultivation of cells and the production of cell therapy products, even in a small clean room.

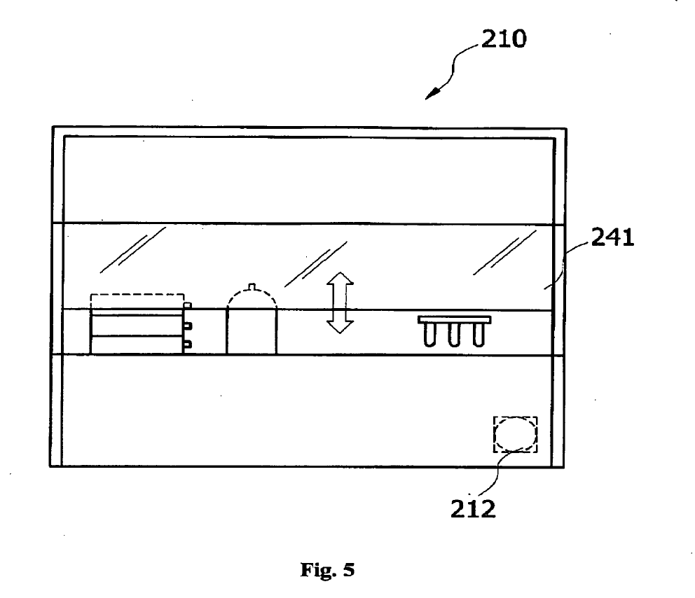

As shown in FIG. 5, the first clean bench 210 includes a device (not shown) for washing and sterilizing a rod used to suction a cell cultivation solution, a vacuum pump 212 provided in a lower end thereof for operating the cell cultivation solution suction rod and the washing and sterilizing device, a first foot switch 213 connected to the vacuum pump for selectively operating the vacuum pump, and a foldable drawer 211 provided in a lower end of a front side thereof for receiving consumption goods having a high use frequency.

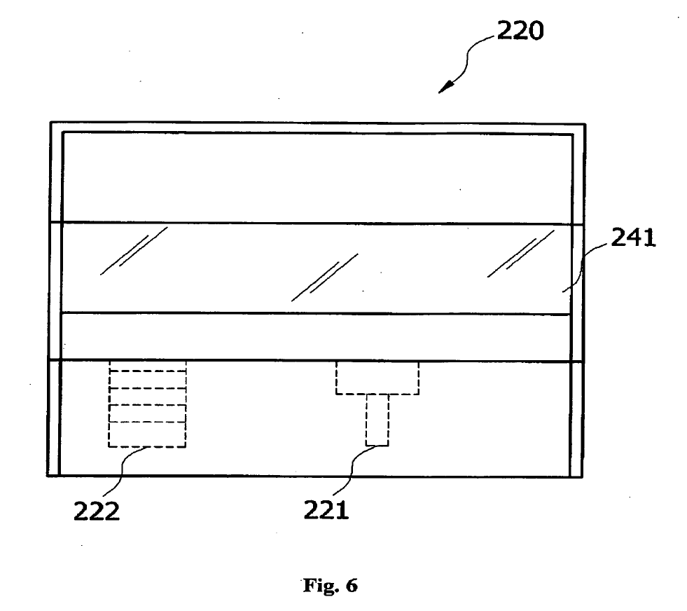

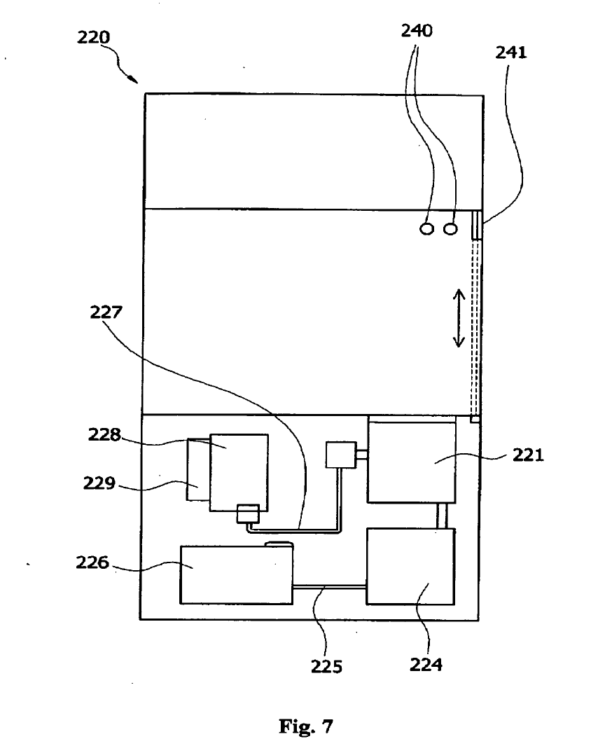

As shown in FIGS. 6 and 7, the second clean bench 220 includes a centrifugal machine 222 and a constant-temperature water bath 221. A waste water pump 224, a waste water pipe 225, and a waste water vessel 226 are installed in sequence below the constant-temperature water bath. In addition, a distilled water inlet pipe 227, a distilled water storage container 228, and a distilled water generator 229 are installed in sequence at a side of the constant-temperature water bath.

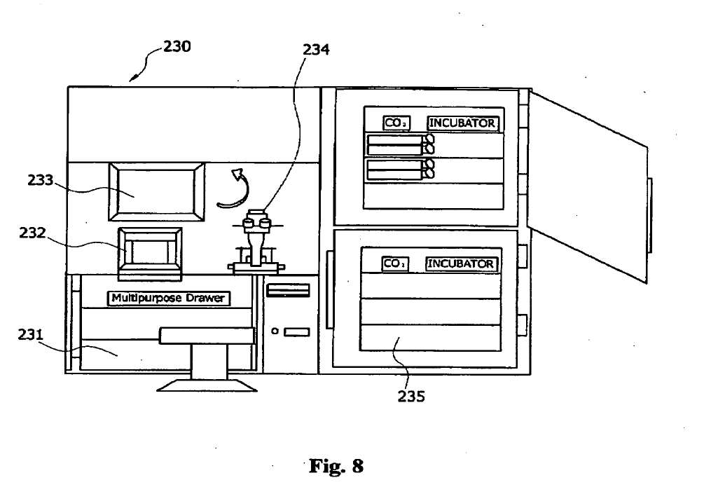

As shown in FIG. 8, the third clean bench 230 includes a multipurpose drawer chest 231 provided in a lower end of the left region thereof, an electronic scale 232 and a microscope 234 placed on the multipurpose drawer chest, a monitor 233 installed in the ceiling thereof, and a carbon dioxide incubator 235 provided in a lower end of the right region thereof, which are arranged to maximize the utility of an operating space.

As shown in FIG. 4, a fluorescent light and UV sterilizing light 240 are installed in a front position of the ceiling of each of the first, second, and third clean benches 210, 220, and 230, to reduce the fatigue of the operator's eyes by effulgence.

A transparent plate 241 is installed to a front side of each of the first, second, and third clean benches 210, 220, and 230. The transparent plate 241 is adjustable in height by selective operation of a second foot switch 214.

In the present invention, a curtain 242 is provided at a lower end of the outer wall surface of the partition defining a clean room in which the U-shaped clean benches are received, to prevent the backflow of outside air when the operation of an air conditioner stops.

In the present invention, furthermore, as shown in FIG. 4, all the first, second, and third clean benches 210, 220, and 230 are provided, at their lower ends, with a plurality of casters 243 by a constant interval, to facilitate movement of the clean benches.

Meanwhile, it is noted that the above described configuration of the present invention may be changed in various manners to have other different shapes.

It is also noted that the present invention is not limited to the above mentioned specific configuration and ma include various modifications, equivalents and substitutions within the spirit and scope of the present invention as defined by the accompanying claims.

Now, the operation and effects of the cell manipulation and cultivation equipment for the production of cell therapy products having the above described configuration acceding to the present invention will be described.

First, in the cell manipulation and cultivation equipment for the production of cell therapy products according to the present invention, a variety of tools and utensils are integrally formed and arranged to achieve the operator's optimum movement path, so as to facilitate the construction of a clean room even in a small laboratory or building. This will be described in more detail hereinafter.

As shown in FIG. 3, the operator is able to access the interior of the room 100 for the production and testing of cell therapy products through the outer entrance/exit door 102, and to access the interior of the clean bench region inside the partition 103 through the inner entrance/exit door 104. In this case, by placing the freezer 105, refrigerator 106 and liquid nitrogen tank 107 in the space 101 outside of the clean bench region, the present invention has the effects of reducing the size of the clean bench region and preventing heat emitted from the freezer and refrigerator from entering the clean bench device 200.

The first, second, and third clean benches 210, 220, and 230 of the clean bench device 200 have a U-shaped arrangement, to increase the overall space of the clean benches, thereby achieving a sufficient operating space required for the cultivation of cells and the production of cell therapy products even in a small clean room.

By dividing the clean bench device into the first, second, and third clean benches 210, 220, and 230 and positioning equipment and tools required for the cultivation of cells in the right place, it is possible to reduce the operator's unnecessary movement and to separately perform an operation per each clean bench according to the possibility of contamination of each clean bench, thereby reducing the possibility of contamination of cells and remarkably improving the clean room and the clean benches.

More specifically, the first clean bench 210 is integrally installed, in the lower end thereof, with the vacuum pump 212 such that the vacuum pump 212 can be operated by the first foot switch 213. In particular, the first clean bench is installed with the device (not shown) for washing and sterilizing the cell cultivation solution suction rod, to eliminate the troublesome exchange of the suction rod.

The second clean bench 220 is integrally installed with the constant-temperature water bath 221 and the centrifugal machine 222, for shortening the operator's movement path during an operation while allowing the implementation of an operation requiring frequent introduction of contaminants when the second clean bench 220 is not used.

An operating panel 223 is provided on the second clean bench for allowing lids of the constant-temperature water bath and centrifugal machine to be electrically operated, so as to achieve convenient input of commands and operation of the tools.

The second clean bench 220 is further provided with the drain device 224, 225 and 226 for the constant-temperature water bath 221 for facilitating drainage of the used cultivation solution and with the distilled water device 227, 228 and 229 for facilitating the supplement of distilled water.

The third clean bench 230 is integrally installed with the microscope 234, computer monitor 223 and carbon dioxide incubator 235. In particular, the electronic scale 232 is installed on an operating table of the third clean bench provided with the monitor, for the efficient use of an operating space. The operating table is also provided with a keypad, for allowing the operator to easily input various information, such as the number of cells and other measured values, while performing an operation. In addition, the computer monitor is connected to a digital camera (not shown) attached to the microscope, to allow the operator to observe the progress of the operation at a real time.

Each of the first, second, and third clean benches 210, 220, and 230 is installed in the front position of the ceiling thereof with the fluorescent light and ultraviolet light 240, so as to reduce the fatigue of the operator's eyes by effulgence.

The transparent plate 241, which is installed in the front side of each of the first, second, and third clean benches 210, 220, and 230, is adjustable in height by the second foot switch 214.

The curtain 242 is installed at the lower end of the outer wall surface of the partition 103 defining the clean bench region in which the U-shaped clean bench device 200 is received, so as to prevent the backflow of outside air when the operation of the air conditioner stops.

The first, second, and third clean benches 210, 220, and 230 are provided, at their lower ends, with the casters 243, so as to facilitate the movement and structural change of the clean benches.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1-10. (canceled)

11. Cell manipulation and cultivation equipment for the production of cell therapy products comprising:
a room for the production and testing of cell therapy product, the room having an inner space enclosed in all sides and an outer entrance/exit door provided at any one side thereof,
an L-shaped partition installed in a position of the room for the production and testing of cell therapy products, the partition having an inner entrance/exit door provided at any one side thereof, and
a clean bench device placed inside the partition, the clean bench device including first, second, and third clean benches for preventing contamination of cells and increasing the utility of a space to the maximum extent.

12. The equipment according to claim 11, further comprising:
a freezer, a refrigerator, and a liquid nitrogen tank placed in the space of the room outside of the clean bench device, to prevent heat emitted from the freezer and refrigerator from entering the clean benches and to reduce the size of the clean benches.

13. The equipment according to claim 11, wherein the first, second, and third clean benches have a U-shaped arrangement such that the second and third clean benches are located at the left and right sides of the first clean bench, to increase the overall space of the clean benches, thereby achieving a sufficient operating space, required for the cultivation of cells and the production of cell therapy products, even in a small clean room.

14. The equipment according to claim 11, wherein the first clean bench comprises:
a vacuum pump provided in a lower end thereof for a device for aspirating the cell cultivation solution with suction rod,
a first foot switch connected to the vacuum pump for selectively operating the vacuum pump, and
a foldable drawer provided in a lower end of a front side thereof for receiving consumption goods having a high use frequency.

15. The equipment according to claim 11, wherein the second clean bench comprises:
a centrifugal machine,
a constant-temperature water bath,
a waste water pump, a waste water pipe, and a waste water vessel installed in sequence below the constant-temperature water bath, and
a distilled water inlet pipe, a distilled water storage container, and a distilled water generator installed in sequence at a side of the constant-temperature water bath.

16. The equipment according to claim 11, wherein the third clean bench comprises:
a multipurpose drawer chest provided in a lower end of one-side region thereof,
an electronic scale and a microscope placed on the multipurpose drawer chest,
a monitor installed in the ceiling thereof, and
a carbon dioxide incubator provided in a lower end of the other side region thereof,
wherein the multipurpose drawer chest, electronic scale, microscope, monitor, and carbon dioxide incubator are arranged to maximize the utility of an operating space.

17. The equipment according to claim 11, wherein a fluorescent light and an ultraviolet (UV) sterilizing light are installed in a front position of the ceiling of each of the first, second, and third clean benches, to reduce the fatigue of the operator's eyes by effulgence.

18. The equipment according to claim 11, wherein a transparent plate is installed to a front side of each of the first second, and third clean benches, the transparent plate being adjustable in height by selective operation of a second foot switch.

19. The equipment according to claim 11, wherein a curtain is provided at a lower end of the outer wall surface of the partition, to prevent the backflow of outside air when the operation of an air conditioner stops.

20. The equipment according to claim 11, wherein a plurality of casters are provided, by a constant interval, at an outer lower end of the partition and a lower end of the clean bench device, to facilitate movement of the partition and the clean benches.
